(19) United States
(12) Patent Application Publication
(10) Pub. No.: US 2023/0358616 A1
(43) Pub. Date: Nov. 9, 2023

(54) Measurement Device

(71) Applicant: Nippon Telegraph and Telephone Corporation, Tokyo (JP)

(72) Inventors: Yujiro Tanaka, Musashino-shi, Tokyo (JP); Daichi Matsunaga, Musashino-shi, Tokyo (JP)

(21) Appl. No.: 18/245,864

(22) PCT Filed: Sep. 25, 2020
(86) PCT No.: PCT/JP2020/036215

§ 371 Date: Mar. 17, 2023

(51) Int. Cl. G01K 1/16; G01K 7/16; G01K 13/20

(52) U.S. Cl. CPC G01K 1/16; G01K 7/16; G01K 13/20; A61B 5/01

(57) ABSTRACT

A measurement device includes: a first member in which a thermal resistor having a temperature sensor for measuring a heat flux transported from a measurement target is installed; a second member that is disposed in a housing including a bottom frame and a side frame of the first member and suppresses an influence of disturbance of outside air on the temperature sensor due to outside air flow or a change in temperature; and a third member that seals an upper surface portion of the first member. The measurement device has an internal structure that suppresses the influence of the disturbance of the outside air on the thermal resistor, and can accurately estimate the core temperature of the measurement target.

### TECHNICAL FIELD

The present invention relates to a measurement device for measuring a core temperature of a measurement target such as a living body.

### BACKGROUND ART

Conventionally, a technique for non-invasively measuring a core temperature of a living body is known. For example, Patent Literature 1 discloses a technique for estimating a core temperature of a living body by assuming a pseudo one-dimensional model in a living body B, a measuring instrument 50 including a temperature sensor and a heat flux sensor, and outside air.

In the technique disclosed in Patent Literature 1, a core temperature of a living body is estimated by assuming a one-dimensional model of biological heat transfer illustrated in FIG. 8. Tair is the temperature of the outside air, Tbody is the core temperature of the living body B, Hsignal is the heat flux flowing into the sensor of the measuring instrument 50, Rsensor is the thermal resistance of the sensor of the measuring instrument 50, Rbody is the thermal resistance of the living body B, Rair is the thermal resistance when the heat flux Hsignal moves to the outside air, Tskin is the temperature of the contact point between the temperature sensor disposed on the skin SK and the skin SK of the living body B, and Ttop is the temperature of the disposition position of the upper temperature sensor.

In Patent Literature 1, a core temperature of a living body is estimated from the following relational expression (1).

Core temperature(Tbody)=temperature(Tskin) of contact point between temperature sensor and skin+coefficient of proportionality A×heat flux(Hsignal)flowing into temperature sensor (1)

The coefficient of proportionality A can be generally obtained by giving a rectal temperature or an eardrum temperature measured using a sensor such as another temperature sensor as a core temperature (Tbody), and thus the core temperature of the living body can be estimated by measuring a heat flux (Hsignal) flowing into the temperature sensor.

### CITATION LIST

### Patent Literature

Patent Literature 1: JP 2020-003291 A

### SUMMARY OF INVENTION

### Technical Problem

However, in a case where a one-dimensional model is assumed as a heat transfer model of a living body as in Patent Literature 1, when outside air is disturbed due to generation of wind or the like and heat flows into the sensor from the outside air, a part of the heat flux Hsignal that should originally flow into the sensor deviates from the sensor. Thus, it becomes impossible to measure the core temperature accurately. Therefore, it is desirable to have a structure for suppressing an influence of disturbance of outside air as a configuration of the measurement device for measuring the core temperature.

The present invention has been made to solve the above-described problems, and an object of the present invention is to provide a measurement device having a structure capable of suppressing an influence of disturbance of outside air and accurately measuring a core temperature.

### Solution to Problem

In order to solve the above-described problems, according to the present invention, there is provided a measurement device including: a thermal resistor in which a measuring instrument for measuring a heat flux transported from a measurement target is installed; a first member including a bottom frame and a side frame in which the thermal resistor is installed at a predetermined position; a second member that is disposed in a housing including the bottom frame and the side frame of the first member, has a shape covering the thermal resistor, and is made of a material having thermal conductivity; and a third member that seals an upper surface portion of the housing in which the second member is disposed, in which the second member is positioned with respect to the housing by fitting a protrusion or a protruding piece provided in the second member into a through hole provided in the bottom frame of the first member, and the protrusion or the protruding piece is exposed to the outside of the bottom frame through the through hole and is configured to be able to contact the measurement target.

### Advantageous Effects of Invention

According to the present invention, it is possible to provide a measurement device having a structure capable of suppressing an influence of disturbance of outside air and accurately measuring a core temperature.

### BRIEF DESCRIPTION OF DRAWINGS

FIG. 1A is a view illustrating an example of a top view of a first member of a measurement device according to an embodiment of the present invention.

FIG. 1B is a view illustrating an example of a side view of the first member of the measurement device according to the embodiment of the present invention.

FIG. 2A is a view illustrating an example of a top view of a second member of the measurement device according to the embodiment of the present invention.

FIG. 2B is a view illustrating an example of a side view of the second member of the measurement device according to the embodiment of the present invention.

FIG. 2C is a view illustrating another example of a side view of the second member of the measurement device according to the embodiment of the present invention.

FIG. 2D is a view illustrating another example of a side view of the second member of the measurement device according to the embodiment of the present invention.

FIG. 2E is a view illustrating another example of a side view of the second member of the measurement device according to the embodiment of the present invention.

FIG. 2F is a view illustrating another example of a side view of the second member of the measurement device according to the embodiment of the present invention.

FIG. 3A is a view illustrating an example of a top view of a third member of the measurement device according to the embodiment of the present invention.

FIG. 3B is a view illustrating an example of a side view of the third member of the measurement device according to the embodiment of the present invention.

FIG. 4A is a view illustrating an example of a side view of the measurement device according to the embodiment of the present invention.

FIG. 4B is a view illustrating an example of dimensions of the measurement device according to the embodiment of the present invention.

FIG. 5 is a view illustrating an example of a cross-sectional view of the measurement device according to the embodiment of the present invention.

FIG. 6 is an estimation result of a core temperature in the measurement device according to the embodiment of the present invention.

FIG. 7 is an example of a block diagram of the measurement device according to the embodiment of the present invention.

FIG. 8 is a heat equivalent circuit for estimating a core temperature by a heat flux.

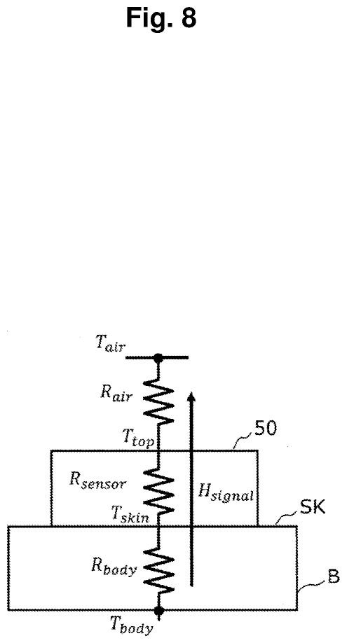

### DESCRIPTION OF EMBODIMENTS

Hereinafter, a preferred embodiment of the present invention will be described. In the following embodiment, a measurement target is a living body, and a measurement surface on which a measurement device is disposed is a surface of the skin of a living body that is the measurement target.

### Outline of Embodiment of Present Invention

A measurement device of the present invention includes: a first member in which a thermal resistor in which a temperature sensor that is a measuring instrument for measuring a heat flux transported from a measurement target is installed is installed; a second member that is disposed in a housing including a bottom frame and a side frame of the first member and suppresses an influence of disturbance of heat transfer to the measuring instrument due to outside air flow or a change in temperature; and a third member that seals an upper surface portion of the first member.

The bottom frame of the first member is provided with a through hole, and the second member is provided with a protrusion or a protruding piece for fitting into the through hole 13 of the first member. By fitting the protrusion or the protruding piece provided in the second member into the through hole provided in the bottom frame of the first member, the second member is positioned with respect to a housing including the bottom frame and the side frame of the first member, whereby the relative position of the second member 20 with respect to the first member can be fixed.

The protrusion or the protruding piece of the second member is exposed to the outside of the bottom frame through the through hole of the first member, and at least a part of the protrusion or the protruding piece is configured to be able to contact the measurement target. Thus, an internal structure for transporting a heat flux from a measurement target outside the thermal resistor to the upper portion of the thermal resistor and suppressing the influence of disturbance of the outside air on the thermal resistor can be configured.

According to the measurement device of the present embodiment, the housing of the measurement device, the internal structure for suppressing the influence of the disturbance of the outside air, and the thermal resistor in which the temperature sensor is installed can constitute the measurement device having the structure relatively positioned and fixed by the combination of the members having a simple structure, and the measurement device having a structure capable of suppressing the influence of the disturbance of the outside air and accurately measuring the core temperature can be provided.

In addition, if the first member constituting the housing of the measurement device and the thermal resistor including the sensor for estimating the core temperature are integrally molded, the relative position between the housing and the thermal resistor can be fixed.

Furthermore, if the thermal resistor is configured to include a hole for installing the temperature sensor at a predetermined position of the thermal resistor, the relative position between the temperature sensor of the thermal resistor and the housing can be fixed.

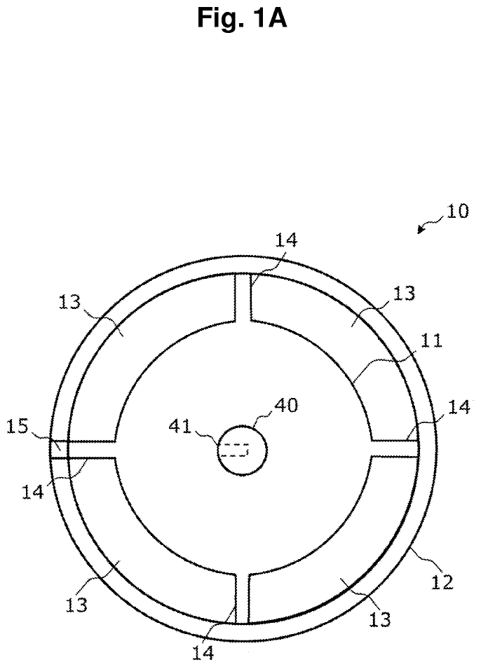

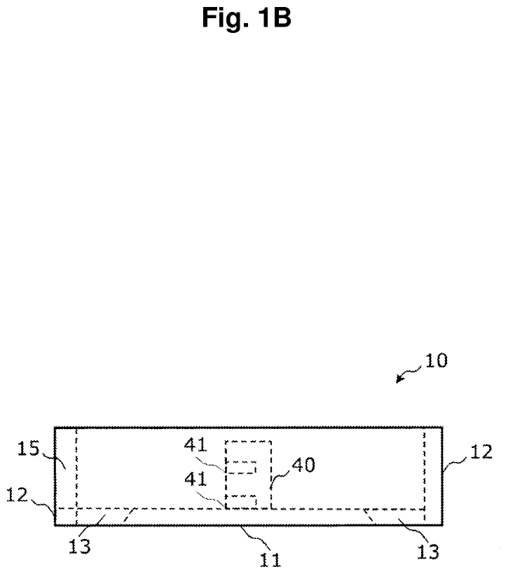

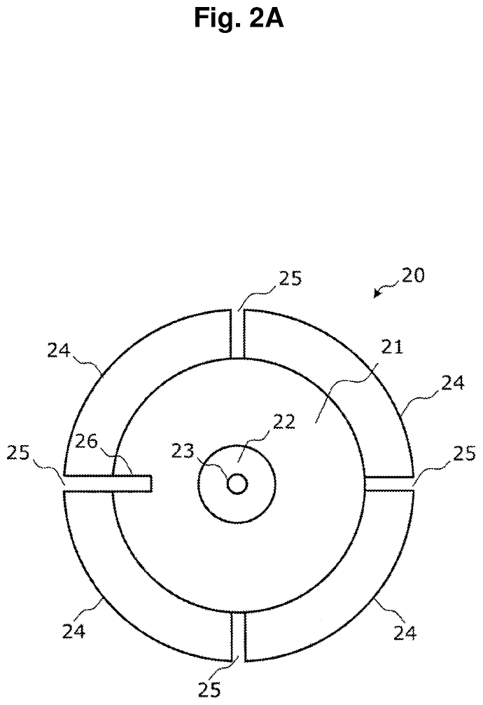

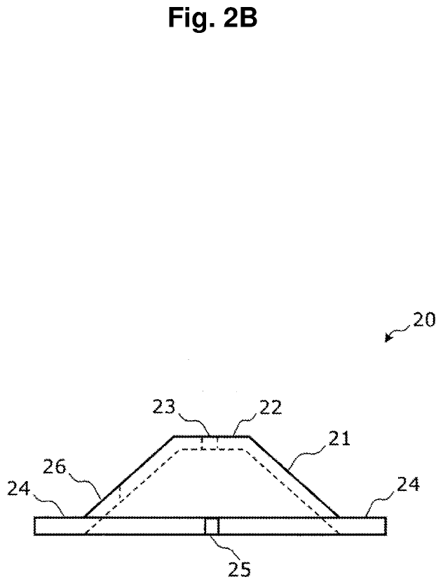

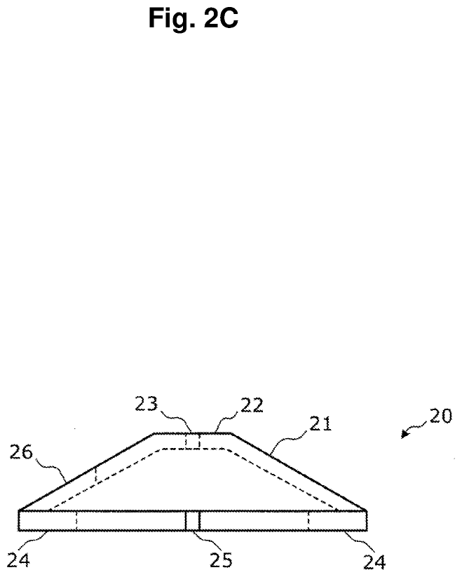

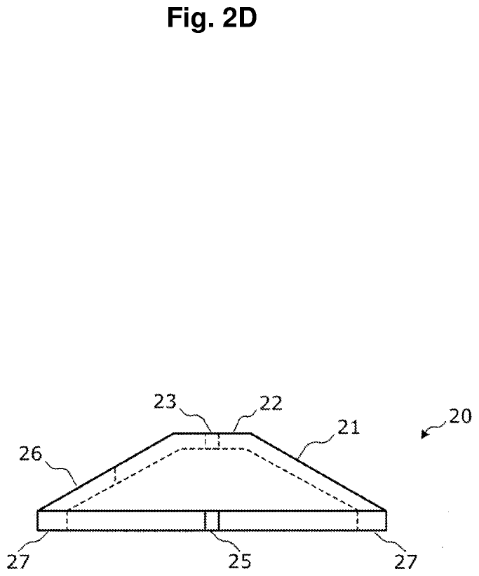

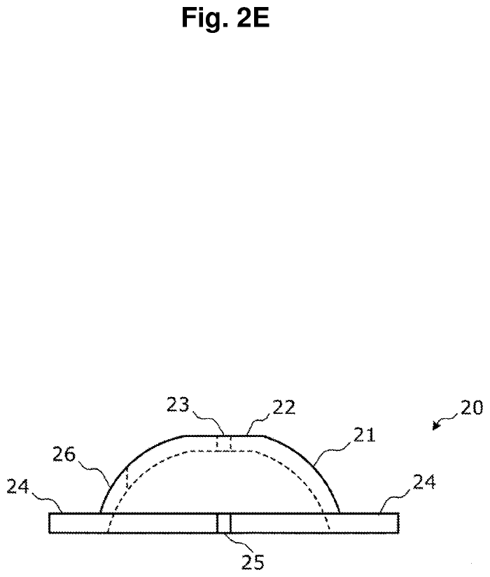

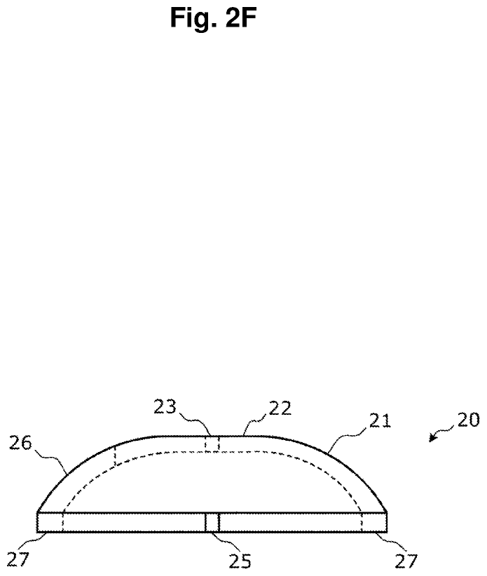

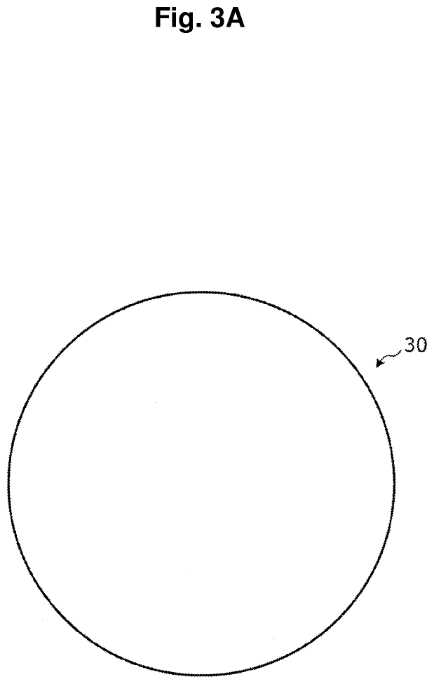

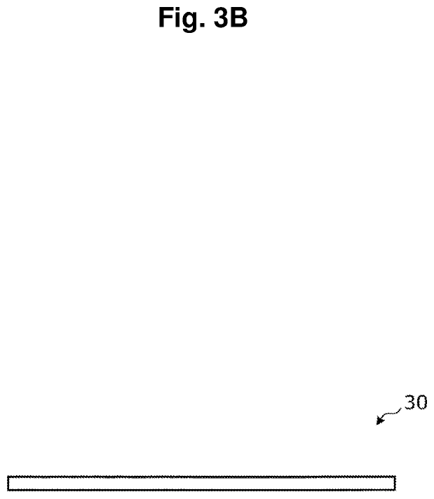

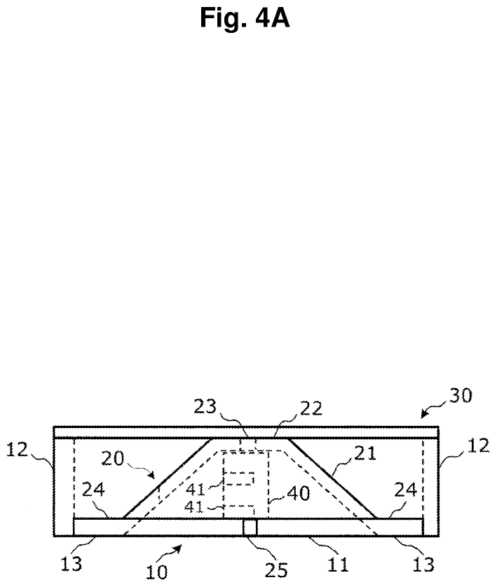

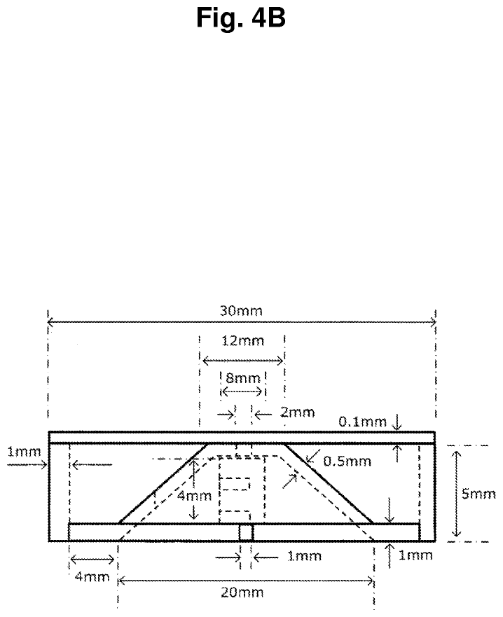

Hereinafter, specific examples of members constituting the measurement device of the present embodiment will be described. FIGS. 1A and 1B are configuration examples of a first member 10, FIGS. 2A to 2F are configuration examples of a second member 20, and FIGS. 3A and 3B are configuration examples of a third member 30. The first member 10, the second member 20, and the third member 30 which will be described below are combined to form the structure of the measurement device of FIG. 4A.

### First Member 10

FIGS. 1A and 1B are configuration examples of first member 10. The first member 10 includes a bottom frame 11 for installing a thermal resistor 40 and a side frame 12 for protecting a sensor and the like of the thermal resistor 40. The bottom frame 11 and the side frame 12 of the first member 10 constitute a housing of the measurement device.

The first member 10 is made of a material having a heat capacity smaller than that of the skin of a living body that is a measurement target. For example, it can be made of a material such as polyethylene terephthalate (PET) or acrylonitrile butadiene styrene (ABS).

Note that the outer shape of the first member 10 is not limited to the cylindrical shape illustrated in FIGS. 1A and 1B, and may be, for example, a prismatic shape or the like. The outer shapes of the thermal resistor 40, the second member 20, and the third member 30 can also employ a configuration of various shapes to correspond to the shape of the first member.

On the bottom frame 11 of the first member 10, the thermal resistor 40 in which a temperature sensor for estimating the core temperature of the living body is installed is integrally molded at a predetermined position. The thermal resistor 40 is made of a material having a predetermined thermal resistance. The thermal resistor 40 includes a hole 41 for fixing a temperature sensor at a predetermined position of the thermal resistor 40. By inserting the temperature sensor into the hole 41, the temperature sensor can be fixed at a predetermined position of the thermal resistor 40.

The hole 41 for fixing the temperature sensor of the thermal resistor 40 has a depth such that a temperature measurement unit of the temperature sensor is disposed at the center of the thermal resistor 40. The cross-sectional view of the hole 41 may be configured to have a cross section whose width decreases toward the lower side of the thermal resistor, that is, toward the bottom frame 11. With such a configuration, the temperature measurement unit of the temperature sensor can be disposed at the center of the thermal resistor 40.

The bottom frame 11 of the first member 10 is provided with through holes 13 into which protruding pieces 24 of the second member 20 are fitted. The bottom frame 11 of the first member 10 is coupled to the side frame 12 by a beam 14 disposed between the through holes 13. By fitting the protruding piece 24 provided in the second member 20 into the through hole 13, the relative position of the second member 20 in the radial direction with respect to the housing of the first member 10 is positioned.

The shape of the through hole 13 is determined according to the shape of the protruding piece 24 to be fitted. In the configuration example of FIG. 1A, the shape of the through hole 13 also has a fan shape corresponding to the shape of the protruding piece 24 having a fan shape. The protruding piece 24 of the second member 20 fitted into the through hole 13 is exposed to the outside of the bottom frame 11 through the through hole 13 of the first member 10, and at least a part of the protruding piece 24 is configured to be able to contact the measurement target through the through hole 13.

FIG. 1A illustrates a configuration in which four through holes 13 having a fan shape are provided in the bottom frame 11 of the first member 10. The shape, size, and number of the through holes 13 are not limited to those illustrated in FIG. 1A, and various shapes, sizes, and numbers can be employed as long as the functions and effects of the present invention are exhibited. For example, the radial length of the fan shape may be changed, or the circumferential length of the fan shape may be changed, and the width of the beam 14 between the through holes 13 may be changed accordingly.

The side frame 12 of the first member 10 may be provided with a wiring slit 15 for drawing electrical wiring to the outside of the housing when a wired sensor such as a thermistor or a resistance temperature detector is used as the temperature sensor of the thermal resistor 40.

### Second Member 20

FIGS. 2A and 2B are configuration examples of the second member 20. The second member 20 is disposed in the housing including the bottom frame 11 and the side frame 12 of the first member 10, and has an internal structure for preventing the influence of disturbance of the outside air due to outside air flow or a change in temperature from being given to the temperature sensor of the thermal resistor 40. The second member 20 has a shape covering the thermal resistor 40 installed at a predetermined position of the first member 10, and is made of a material having thermal conductivity, for example, aluminum.

The shape of the second member 20 is determined according to the shape of the housing of the first member 10. The second member 20 in FIG. 2A has a circular shape corresponding to the shape in FIG. 1A. In the configuration examples of FIGS. 2A and 2B, the second member 20 has a truncated cone shape for covering the thermal resistor 40 in a part thereof, and four protruding pieces 24 having a fan shape toward the outside of a tapered portion 21 having the truncated cone shape are formed at a circular edge of the tapered portion 21. The shape of the protruding piece 24 is determined according to the shape of the through hole 13 to be fitted. In the configuration example of FIG. 2A, the protruding piece 24 also has a fan shape corresponding to the shape of the through hole 13 having a fan shape.

In the configuration example of FIG. 2A, a slit 25 having a penetrating structure is formed between the protruding pieces 24 such that the beam 14 of the first member 10 is fitted. As the structure between the protruding pieces 24, other configurations can be employed as long as the beam 14 of the first member 10 is fitted. For example, the first member 10 may be formed of a groove having no penetrating structure into which the beam 14 of the first member 10 is fitted.

A truncated cone 22 having a truncated cone shape of the second member 20 may be provided with a hole 23 penetrating the second member 20. By appropriately adjusting the size of the hole 23 penetrating the second member 20, it is possible to adjust the depth to be estimated in a case of estimating the core temperature of the living body.

The second member 20 may be provided with a wiring slit 26 for drawing electrical wiring to the outside of the housing when a wired temperature sensor such as a thermistor or a resistance temperature detector is used as the temperature sensor.

The configuration of the protruding piece 24 of the second member 20 is not limited to the configurations of FIGS. 2A and 2B. For example, as illustrated in FIG. 2C, a configuration may be employed in which a plurality of protruding pieces 24 directed toward the inside of the tapered portion 21 are formed at a circular edge of the tapered portion 21 having a truncated cone shape.

As illustrated in FIG. 2D, the entire second member 20 may be formed in a truncated cone shape, a protrusion 27 may be formed in an edge of the tapered portion 21 having the truncated cone shape toward the bottom frame 11 of the first member 10, and the protrusion 27 may be recessed into the through hole 13 of the bottom frame 11. In this case, the through hole 13 of the first member 10 is configured to have a shape and a dimension corresponding to the protrusion 27.

The shape of the second member 20 is not limited to the truncated cone shape as long as it can exhibit the function of covering the thermal resistor 40, and various shapes such as other cone shapes and frustum shapes can be employed. For example, as illustrated in FIGS. 2E and 2F, a dome shape or a spherical shape may be employed as the shape of the second member 20, and the thermal resistor 40 may be disposed inside the dome shape or the spherical shape, or a pyramid shape configuration may be employed when the first member has a prismatic shape.

In the second member 20, the protruding piece 24 is fitted into the through hole 13 of the first member 10, the relative position of the second member 20 in the radial direction with respect to the housing of the first member 10 is positioned, and the second member is exposed to the outside of the bottom frame 11 through the through hole 13, so that the second member can be brought into thermal contact with the measurement target. With such a configuration, an internal structure for transporting a heat flux from a measurement target outside the thermal resistor 40 to the upper portion of the thermal resistor 40 and suppressing the influence of disturbance of the outside air on the temperature sensor of the thermal resistor 40 can be configured.

FIG. 2A illustrates a configuration in which four protruding pieces 24 having a fan shape are provided in the bottom frame 11 of the second member 20. The shape, size, and number of the protruding pieces 24 are not limited to those illustrated in FIG. 2A, and various shapes, sizes, and numbers can be employed as long as the functions and effects of the present invention are exhibited. For example, the radial length of the fan shape may be changed, or the circumferential length may be changed, and the width of the slit between the protruding pieces 24 may be changed accordingly.

### Third Member 30

FIGS. 3A and 3B are configuration examples of the third member 30. The third member 30 seals the upper surface portion of the housing of the first member 10 in which the second member 20 is disposed, and has a sheet-like structure. The shape of the third member 30 is determined according to the shape of the housing of the first member 10. The third member 30 in FIG. 3A has a circular shape corresponding to the cylindrical shape of the first member 10 in FIG. 1A. Similarly to the first member 10, the third member 30 is made of a material having a heat capacity smaller than that of the skin of the living body that is a measurement target, and can be made of, for example, a material such as polyethylene terephthalate (PET) or acrylonitrile butadiene styrene (ABS).

### Structure of Measurement Device

FIG. 4A is a configuration example of a structure of the measurement device configured by combining the first member 10 in FIGS. 1A and 1B, the second member 20 in FIGS. 2A to 2B, and the third member 30 in FIGS. 3A and 3B.

According to the measurement device of the present embodiment, the housing of the measurement device, the internal structure for suppressing the influence of the disturbance of the outside air, and the thermal resistor in which the temperature sensor is installed can constitute the measurement device having the structure relatively positioned and fixed by the combination of the members having a simple structure, and the measurement device having a structure capable of suppressing the influence of the disturbance of the outside air and accurately measuring the core temperature can be provided.

FIG. 4B is a view illustrating an example of dimensions of a side view of the measurement device according to the embodiment of the present invention. FIG. 4B illustrates an example of dimensions of the measurement device, and the configuration of the measurement device of the present invention is not intended to be limited to the numerical values illustrated in FIG. 4B.

The first member 10 has a cylindrical shape, the bottom frame 11 has a thickness of 1 mm, and the side frame 12 has an outer diameter of 30 mm and a thickness of 1 mm. The through hole 13 provided in the bottom frame 11 has a radial length of 4 mm, and the beam 14 between the through holes 13 has a width of 1 mm and a length of 4 mm. The thermal resistor 40 has a cylindrical shape, and has a height of 4 mm and an outer diameter of 8 mm when viewed from the bottom frame 11 in contact with the measurement target.

The second member 20 has a truncated cone shape, the truncated cone 22 has a height of 5 mm and an outer diameter of a dimension corresponding to the outer shape of the first member 10 on which the second member 20 is disposed. The outer diameter of the upper surface portion of the truncated cone 22 is 12 mm, and the thickness of the tapered portion 21 having a truncated cone shape is 0.5 mm. The outer shape of the hole 23 provided in the truncated cone 22 is 2 mm. The protruding piece 24 formed on the circular edge of the tapered portion 21 having a truncated cone shape has a shape and a dimension corresponding to the through hole 13 of the first member 10.

The third member 30 has a thickness of 0.1 mm and an outer diameter of a shape and a dimension corresponding to the outer diameter of the side frame 12 of the first member 10.

### Configuration of Measurement Device

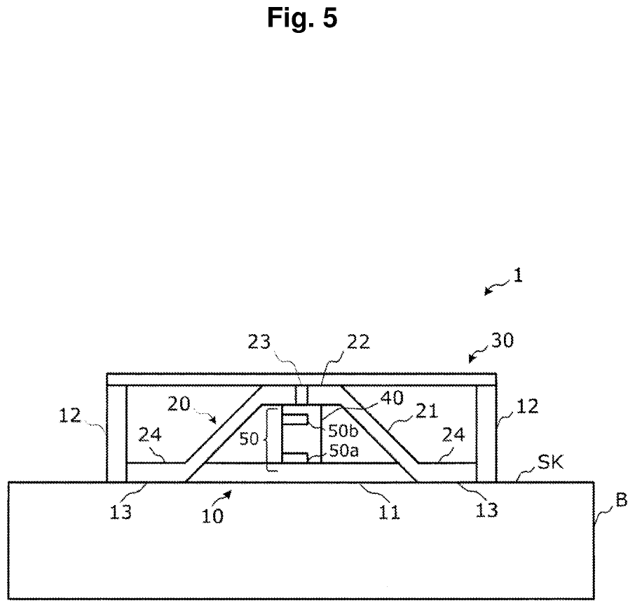

FIG. 5 illustrates an example of a cross-sectional view of the measurement device according to the embodiment of the present invention. FIG. 5 illustrates a cross-sectional view of a measurement device including a thermal resistor 40 including a measuring instrument 50 therein, a first member 10 in which the thermal resistor 40 is installed, a second member covering the thermal resistor 40, and a third member 30 that seals an upper portion of the first member 10.

The measuring instrument 50 disposed inside the thermal resistor 40 includes a sensor for measuring a heat flux transported from a living body B. The second member 20 having a truncated cone shape covering the thermal resistor 40 is configured to transport the heat flux from the living body B outside the first member 10 to an upper surface portion of the thermal resistor 40. Furthermore, although not illustrated in this drawing, the measurement device 1 includes an arithmetic circuit or the like for estimating the core temperature of the living body B using the measurement result of the sensor in addition to the configuration of the measurement device 1 of FIG. 5.

### Configuration of Sensor in Measuring Instrument

The measuring instrument 50 disposed inside the thermal resistor 40 includes a temperature sensor 50a configured to measure the temperature of the skin SK as a measurement surface, and a temperature sensor 50b disposed at a position immediately above the temperature sensor 50a to face the temperature sensor 50a.

In the configuration example of FIG. 5, the heat flux Hsignal is measured using the temperature difference between the temperature Tskin measured by the temperature sensor 50a and the temperature Ttop measured by the temperature sensor 50b. As the temperature sensors 50a and 50b, for example, a thermistor, a thermocouple, a platinum resistor, an IC temperature sensor, or the like can be used.

In the configuration example of FIG. 5, the heat flux Hsignal is measured by the pair of temperature sensors 50a and 50b. However, the temperature Tskin of the measurement surface may be measured by the temperature sensor 50a, and the heat flux Hsignal in the skin SK of the living body B may be measured by the heat flux sensor.

### Estimation Result of Core Temperature

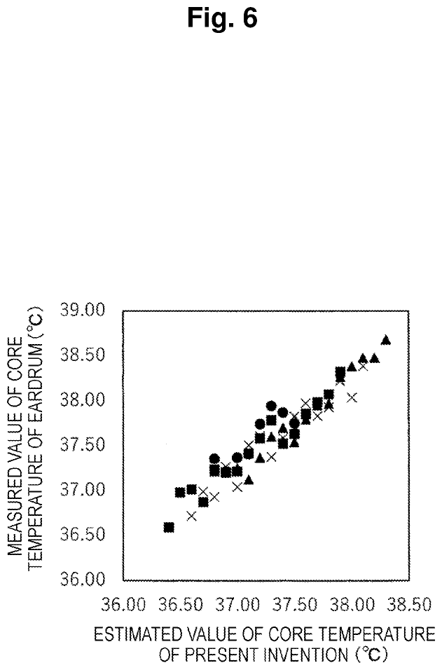

FIG. 6 is an estimation result of the core temperature according to the embodiment of the present invention. FIG. 6 shows a result of comparison between the core temperature estimated using the measurement device of the present embodiment and the measured value of the core temperature measured with the eardrum. According to FIG. 6, with the measurement device of the present embodiment, it can be confirmed that the estimation result of the core temperature with less measurement error of the core temperature is obtained.

### Effect of Present Embodiment

According to the measurement device of the present embodiment, the first member 10 in which the thermal resistor 40 in which the temperature sensor that is a measuring instrument for measuring a heat flux transported from a measurement target is installed is installed, the second member that is disposed in a housing including the bottom frame 11 and the side frame 12 of the first member 10 and suppresses an influence of disturbance of heat transfer due to outside air flow or a change in temperature, and the third member 30 that seals the upper surface portion of the first member 10 are included. The housing of the measurement device, the internal structure for suppressing the influence of the disturbance of the outside air, and the thermal resistor 40 in which the temperature sensor is installed can constitute the measurement device having the structure relatively positioned and fixed by the combination of the members having such a simple structure, and the measurement device having a structure capable of suppressing the influence of the disturbance of the outside air and accurately measuring the core temperature can be provided.

### Configuration Example of Measurement Device

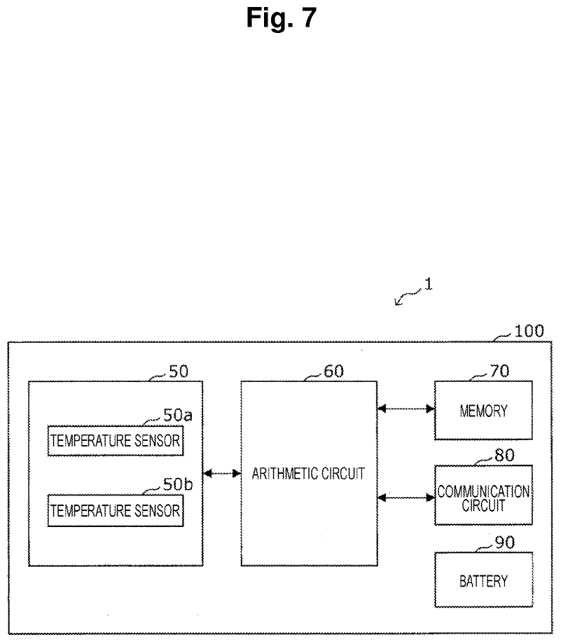

A configuration of the measurement device 1 according to the present embodiment will be described with reference to FIG. 7. As illustrated in FIG. 7, the measurement device 1 includes the configuration of the measurement device 1, an arithmetic circuit 60 that estimates a core temperature, a memory 70, a communication circuit 80, and a battery 90.

The measurement device 1 includes, for example, the measuring instrument 50, the arithmetic circuit 60, the memory 70, the communication circuit 80 that functions as an I/F circuit with the outside, and the battery 90 that supplies power to the arithmetic circuit 60, the communication circuit 80, and the like on a sheet-like base material 100.

The arithmetic circuit 60 calculates an estimated value of the core temperature Tbody from the temperatures Tskin and Ttop measured by the temperature sensors 50a and 50b included in the measuring instrument 50 using Expression (1).

The memory 70 stores the information on the one-dimensional biological heat transfer model based on the above-described Expression (1) and the estimation result of the core temperature. The memory 70 can be realized by a predetermined storage area in a rewritable non-volatile storage device (for example, a flash memory or the like) provided in the measurement system.

The communication circuit 80 outputs the time-series data of the core temperature Tbody of the living body B generated by the arithmetic circuit 60 to the outside. As the communication circuit 80, when data or the like is output by wire, an output circuit to which a USB or other cables can be connected is used, but for example, a wireless communication circuit conforming to Bluetooth (registered trademark), Bluetooth Low Energy, or the like may be used.

The sheet-like base material 100 functions as a base for placing the measurement device 1 including the measuring instrument 50, the arithmetic circuit 60, the memory 70, the communication circuit 80, and the battery 90, and further includes wiring (not illustrated) for electrically connecting these elements. Assuming that the measurement device 1 is connected on the skin of a living body, it is desirable to use a deformable flexible board for the sheet-like base material 100.

Here, the measurement device 1 is realized by a computer. Specifically, the arithmetic circuit 60 is realized by, for example, a processor such as a CPU or a DSP executing various data processing according to a program stored in a storage device, such as a ROM, a RAM, and a flash memory, including the memory 70 provided in the measurement device 1. The program for causing the computer to function as the measurement device 1 can be recorded on a recording medium or provided through a network.

### Modification of Embodiment

Although the embodiments of the measurement device of the present invention have been described above, the present invention is not limited to the described embodiments, and various modifications that can be assumed by those skilled in the art can be made within the scope of the invention described in the claims.

### REFERENCE SIGNS LIST

1 Measurement device
10 First member
11 Bottom frame
12 Side frame
13 Through hole
14 Beam
15 Wiring slit
20 Second member
21 Tapered portion
22 Truncated cone
23 Hole
24 Protruding piece
25 Slit
26 Wiring slit
27 Protrusion
30 Third member
40 Thermal resistor

1. A measurement device comprising:
a thermal resistor in which a measuring instrument for measuring a heat flux transported from a measurement target is installed;
a first member including a bottom frame and a side frame in which the thermal resistor is installed at a predetermined position;
a second member that is disposed in a housing including the bottom frame and the side frame of the first member, has a shape covering the thermal resistor, and is made of a material having thermal conductivity; and
a third member that seals an upper surface portion of the housing in which the second member is disposed,
wherein the second member is positioned with respect to the housing by fitting a protrusion or a protruding piece provided in the second member into a through hole provided in the bottom frame of the first member, and the protrusion or the protruding piece is exposed to an outside of the bottom frame through the through hole and is configured to be able to contact the measurement target.

2. The measurement device according to claim 1, wherein
the thermal resistor is integrally molded at a predetermined position of the bottom frame of the first member.

3. The measurement device according to claim 2, wherein
the thermal resistor includes a hole for installing the measuring instrument at a predetermined position of the thermal resistor.

4. The measurement device according to claim 3, wherein
the hole has a cross section whose width decreases toward the bottom frame in which the thermal resistor is installed.

5. The measurement device according to claim 1, wherein
at least a part of the second member has a cone shape and is configured to transport the heat flux from the measurement target outside the thermal resistor to an upper portion of the thermal resistor.

6. The measurement device according to claim 5, wherein
at least a part of the second member has a frustum shape, and a plurality of the protrusions or protruding pieces are formed at an edge of a tapered portion having the frustum shape.

7. The measurement device according to claim 6, wherein
the thermal resistor has a cylindrical shape, and
at least a part of the second member has a truncated cone shape, and the plurality of protruding pieces having a fan shape are formed at a circular edge of the tapered portion having the truncated cone shape.

8. The measurement device according to claim 7, wherein
the second member includes a hole penetrating the second member in the upper surface portion having the truncated cone shape.

9. The measurement device according to claim 2, wherein
at least a part of the second member has a cone shape and is configured to transport the heat flux from the measurement target outside the thermal resistor to an upper portion of the thermal resistor.

10. The measurement device according to claim 3, wherein
at least a part of the second member has a cone shape and is configured to transport the heat flux from the measurement target outside the thermal resistor to an upper portion of the thermal resistor.

11. The measurement device according to claim 4, wherein
at least a part of the second member has a cone shape and is configured to transport the heat flux from the measurement target outside the thermal resistor to an upper portion of the thermal resistor.
